(19) United States
(12) Patent Application Publication
(10) Pub. No.: US 2008/0056933 A1
(43) Pub. Date: Mar. 6, 2008

(54) Self-Propelled Sterilization Robot and Method

(76) Inventors: Barrett H. Moore, Winnetka, IL (US); Matthew J. Botos, Chicago, IL (US)

Correspondence Address: FITCH EVEN TABIN AND FLANNERY, 120 SOUTH LA SALLE STREET, SUITE 1600, CHICAGO, IL, 60603-3406, US

(21) Appl. No.: 11/468,157

(22) Filed: Aug. 29, 2006

(51) Int. Cl. A61L 2/02

(52) U.S. Cl. 422/1; 250/453.11; 422/124; 96/224; 422/24; 422/4

(57) ABSTRACT

A sterilization apparatus (200) comprises a robot (201), at least one germicidal energy source (202), and at least one motive capability (203). The sterilization apparatus may optionally further comprise numerous additional components, including a filtration unit (204), at least one power source (209), a power connector (210), an environmental sampling device (211), at least one sensor (212), a control system (213), an audio output device (214), a data transmitter (215), a global positioning satellite (GPS) receiver (216), a radio frequency identification (RFID) tag (217), a vacuum device (218), a floor washing device (219), an activator (220), a waterproof housing (221), and/or a padded housing (222).

### TECHNICAL FIELD

This invention relates generally to self-propelled robots.

### BACKGROUND

As powerful as the machinery of modern life appears, modern citizens are today perhaps more at risk of experiencing a serious disruption in their ability to prosper or even to survive en mass than is generally perceived. Genuine concerns exist regarding the threat of harmful airborne chemical and/or biological agents (due, for example, to the detonation of chemical or biological weapons or as may result through inadvertence or accident).

While genuine concerns exist regarding the threat of airborne harmful biological and/or chemical agents due to the purposeful or accidental detonation of a weapon, naturally-occurring microbes also pose a significant threat in our communities, particularly in hospitals. Hospitals face an ongoing battle as microbes, such as Staphylococcus aureus, enterococcus, Pasteurella species, group A streptococci, pneumococcus, Mycobacterium tuberculosis, and Escherichia coli amongst others naturally develop resistance to numerous drugs. The World Health Organization has reported that about 14,000 people are infected and die each year from drug-resistance microbes acquired in U.S. hospitals and that drug-resistant bacteria are responsible for about sixty percent of hospital acquired infections around the world.

Reducing the threat of microbes, particularly drug resistant microbes, is a constant burden on hospitals, schools, day care facilities, veterinary clinics, nursing homes, correctional facilities, barracks, ships, industrial kitchens and/or food preparation facilities, and buildings inhabited by numerous people or animals with a variety of health problems or susceptibilities, such as immunocompromised persons. Compounding these problems, many pathogenic microorganisms, including bacteria, such as Mycobacterium tuberculosis, and viruses, such as smallpox and influenza viruses, may be carried through the air over long distances in small water droplets. Moreover, spore-forming bacteria, such as Bacillus subtilis, and spore-forming fungi, such as Aspergillus fumigatus, can remain airborne essentially indefinitely in air currents and travel throughout a building or other enclosed space. Airborne microbes are extremely difficult to eradicate because they are often able to evade even the most thorough sterilization practices.

### BRIEF DESCRIPTION OF THE DRAWINGS

The above needs are at least partially met through provision of the self-propelled sterilization robot and method described in the following detailed description, particularly when studied in conjunction with the drawings, wherein:

FIG. 1 comprises a flow diagram as configured in accordance with various embodiments of the invention;

FIG. 2 comprises a block diagram as configured in accordance with various embodiments of the invention;

FIG. 3 comprises a schematic front detail view as configured in accordance with various embodiments of the invention;

FIG. 4 comprises a schematic perspective view as configured in accordance with various embodiments of the invention;

FIG. 5 comprises a schematic front detail view as configured in accordance with various embodiments of the invention;

FIG. 6 comprises a schematic side detail view as configured in accordance with various embodiments of the invention;

FIG. 7 comprises a schematic front detail view as configured in accordance with various embodiments of the invention;

FIG. 8 comprises a schematic top detail view as configured in accordance with various embodiments of the invention;

FIG. 9 comprises a schematic front detail view as configured in accordance with various embodiments of the invention;

FIG. 10 comprises a schematic front detail view as configured in accordance with various embodiments of the invention;

FIG. 11 comprises a schematic front detail view as configured in accordance with various embodiments of the invention;

FIG. 12 comprises a block diagram as configured in accordance with various embodiments of the invention;

FIG. 13 comprises a block diagram as configured in accordance with various embodiments of the invention; and

FIG. 14 comprises a block diagram as configured in accordance with various embodiments of the invention.

Skilled artisans will appreciate that elements in the figures are illustrated in a logical representation for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially-feasible embodiment are often not depicted in order to facilitate a less obstructed view of these various embodiments of the present invention. It will further be appreciated that certain actions and/or steps may be described or depicted in a particular order of occurrence while those skilled in the art will understand that such specificity with respect to sequence is not actually required. It will also be understood that the terms and expressions used herein have the ordinary meaning as is accorded to such terms and expressions with respect to their corresponding respective areas of inquiry and study except where specific meanings have otherwise been set forth herein.

### DETAILED DESCRIPTION

Generally speaking, pursuant to these various embodiments, a sterilization apparatus is provided which comprises a robot configured and arranged to move over a surface, at least one motive capability to facilitate movement of the robot over that surface, and a germicidal energy source carried by the robot. The at least one motive capability may comprise wheels, treads, skids, magnets, air propulsion, among others. The germicidal energy source comprises at least one of the group consisting of an ultraviolet (UV) lamp, a radiofrequency electric field (RFEF) apparatus, an electrostatic field apparatus, a heat generating device capable of producing heat at a temperature of at least about 80° C., or a combination thereof. The germicidal energy source may be directed in one or a plurality of directions outward from the robot. By this approach, the germicidal energy source may be aimed towards a surface, such as a floor surface, to destroy or inactivate contaminants, such as microbes, on the surface and/or the germicidal energy source may be aimed toward the ambient air to inactivate airborne contaminants.

By one approach, the robot may further comprise an air filtration unit having at least one filter and at least one air drawer. By one approach the at least one filter in the filtration unit is capable of filtering out airborne particulate matter at least as small as 500 microns. Preferably, for many application settings, the at least one filter in the filtration unit is capable of filtering out airborne particulate matter at least as small as 0.3 microns. The at least one air drawer in the filtration unit is capable of drawing ambient air toward the air filtration unit and through the at least one filter.

The robot may further comprise a variety of sensors to detect conditions that could adversely affect inhabitants of an enclosed space within which the robot operates. By this approach, the robot generates signals in response to such detections so that the robot can implement an appropriate predetermined response.

So configured, such a robot can operate in a partially or fully autonomous manner within a space of interest (such as a hospital operation theater or a civil defense shelter) to neutralize microbes or remove other contaminants that pose a potential risk to human inhabitants of that space. The germicidal energy source, while highly effective to achieve such purposes, tends to represent an approach to which microbes cannot develop resistance over time. As will be shown below in more detail, these teachings are highly flexible and scalable and can readily be applied in a wide variety of application settings. Those skilled in the art will further recognize and appreciate that these teachings are implementable in a cost effective manner.

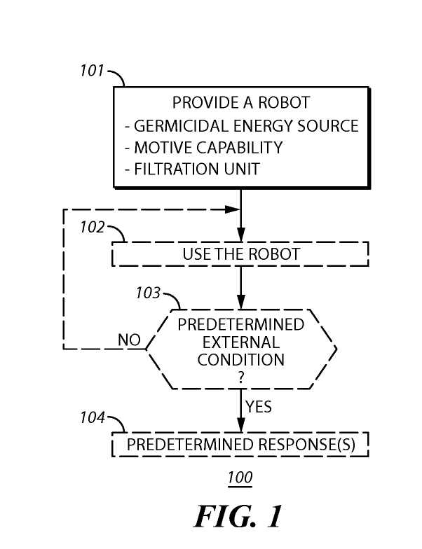

These and other benefits may become clearer upon making a thorough review and study of the following detailed description. Referring now to the drawings and in particular to FIG. 1, a corresponding process 100 accommodates providing 101 a robot, wherein the robot comprises a germicidal energy source, a motive capability, and a filtration unit for filtering the ambient air. The robot may be conveniently used 102 in a variety of buildings or enclosed spaces, such as hospitals, storage facilities, barracks, ships, industrial kitchens and/or food preparation facilities, civil defense shelters, schools, day care facilities, veterinary clinics, nursing homes, correctional facilities, and facilities inhabited or visited by persons or animals with health problems or susceptibilities, among others.

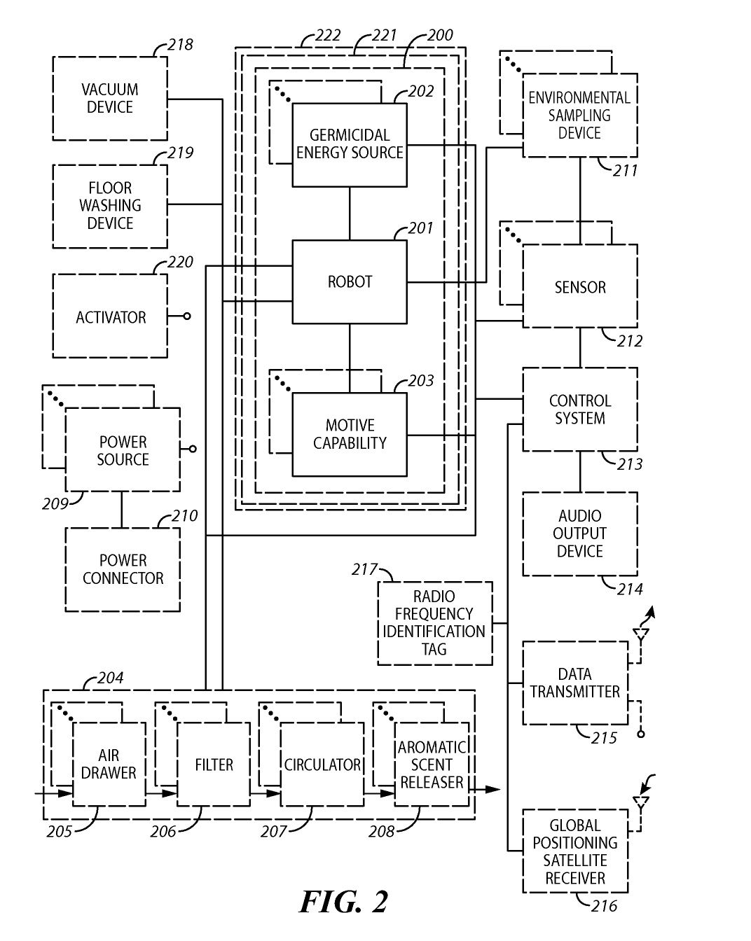

Referring now to FIG. 2, a sterilization apparatus 200 is shown. The sterilization apparatus comprises a robot 201 that is configured and arranged to move over a surface, such as a floor surface.

The robot 201 comprises at least one germicidal energy source 202 selected from the group consisting of ultraviolet (UV) lamps, radiofrequency electric field (RFEF) apparatuses, electrostatic field apparatuses, and heat-generating devices capable of producing heat at temperature of at least about 80° C., or a combination thereof. As used herein, the term “germicidal” means the germicidal energy source is capable of inactivating, inhibiting the growth of, or killing microbes.

The at least one germicidal energy source 202 is generally capable of reducing the number of vegetative and/or spore-forming microbes in an enclosed space. In one aspect, the at least one germicidal energy source is capable of reducing the number of bacteria to acceptable levels, such as levels that pose little health risk to humans or animals. In one aspect, the at least one germicidal energy source is capable of reducing the number of bacteria by at least about a one log reduction. In another aspect, the at least one germicidal energy source is capable of reducing the number of bacteria by at least about a two log reduction. In another aspect, the at least one germicidal energy source is capable of reducing the number of bacteria by at least about a three log reduction. In another aspect, the at least one germicidal energy source is capable of reducing the number of bacteria by at least about a four log reduction. In another aspect, the at least one germicidal energy source is capable of reducing the number of bacteria by at least about a five log reduction.

In addition to being able to inactivate, inhibit the growth of, or kill microbes, the germicidal energy source in some instances may be capable of neutralizing other airborne contaminants, such as allergens. For example, certain allergens, such as animal dander, are comprised of proteins which may be denatured when exposed to high temperatures and the allergenicity may be reduced.

A heat generating device capable of producing heat at a temperature of at least about 80° C. may comprise a variety of heat generating devices. It should be noted that the temperature produced by the heat generating device will determine the residence time for the ambient air necessary for contaminants to be inactivated or killed. The higher the temperature produced by the heat generating device, the shorter the residence time in the heat generating device necessary to be effective for inactivating contaminants in the air. For example, the residence time at 80° C. should be in the range of about 30 to about 120 seconds. At a higher temperature, the residence time may be substantially shorter. In one aspect the heat generating device comprises a heated pipe through which ambient air is drawn by an air drawer. In another aspect, the heat generating device may be a plate heat exchanger or tubular heat exchanger as known in the art.

An RFEF apparatus provides for the non-thermal inactivation of microbes by the application of high-intensity RFEF having electric field strength of up to 20 kilovolts per centimeter and frequencies in the range of 15 to 70 kilohertz. The RFEF apparatus is particularly effective when combined with a heat generating device.

An electrostatic apparatus, such as an electrostatic precipitator, may be used to destroy and/or remove particulate matter, including dust, allergens, and microbes, from the air using an induced electrostatic charge. As known in the art, electrostatic precipitators generally comprise metal collection plates and a high voltage power supply.

UV lamps, such as commercially available mercury-vapor lamps, emit a majority of their light at a wavelength of about 253.7 nanometers (nm) (generally referred to as a 254 nm UV lamp), which is effective to cause defects in microbial deoxyribonucleic acid (DNA), thus rendering the microbes harmless, although not necessarily dead.

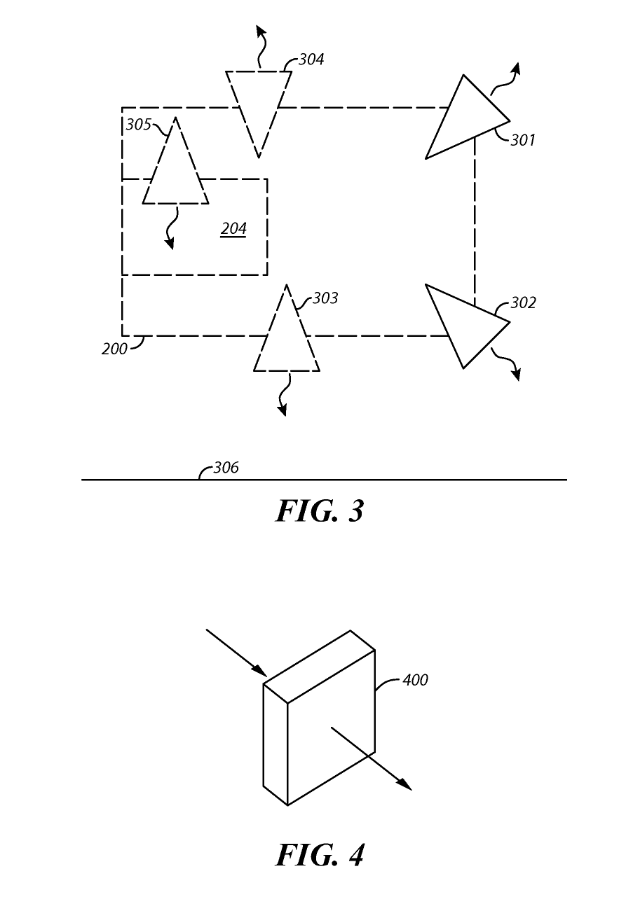

In another aspect of the invention, there may be more than one 254 nm UV lamp. As shown in FIG. 3, multiple UV lamps 301, 302, 303, and 304 are aimed in multiple directions to emit UV radiation outwardly from the robot 201. By one approach, at least one 254 nm UV lamp 301 is configured and arranged to emit UV radiation outwardly from the robot 201 into the ambient air and at least one 254 nm UV lamp 302 is configured and arranged to emit UV radiation outwardly from the robot 201 toward a surface 306, such as a floor surface. By this arrangement, ambient air around the robot and a floor surface are both sterilized (simultaneously, if desired). It should be noted that this surface 306 is not limited to floor surfaces. This surface 306 could comprise a variety of surfaces, including the underside of a table or chair, or the top of a table.

Referring again to FIG. 2, the robot 201 further comprises at least one motive capability 203 that facilitates movement of the robot 201 over a surface, such as a floor surface. Generally, the movement of the robot may be in virtually any direction, such as rotational, lateral, or elevational movement. The robot's directional movement may be random, predetermined, programmed, or directed in real time.

The at least one motive capability 203 may comprise wheels, treads, skids, magnets, air propulsion, or the like, or combinations thereof. In another aspect, the motive capability 203 may comprise an elevational support system, such as a leg, tube, or other extendable device, that elevates the entire robot 201 or any component(s) or combination of components thereof (such as, but not limited to, an air filtration device, the germicidal energy source, a sensor, and so forth) such that the robot 201 is able to increase the zone of sterilization. In another aspect, the motive capability 203 may comprise walking legs powered by compressed air as are known in the art. The walking legs may further comprise suction cups or non-slip materials as are well known in the art to provide traction. In another aspect, the motive capability 203 may comprise one or more horizontal rotors which lift and propel the robot 201 elevationally, rotationally, and/or laterally. In yet another aspect, the robot 201 may be reversibly fixed to an elongated structure, such as a pole, or a series of elongated structures connected to form a track-like system, so that the robot 201 moves rotationally, elevationally, and/or laterally along a predetermined course. This aspect is particularly useful for zero or low gravity application settings.

The robot 201 may further comprise an air filtration unit 204 as shown in FIG. 2 with the arrows indicating the direction of airflow through the air filtration unit. The air filtration unit 204 comprises at least one filter 206 capable of filtering out airborne particulate matter at least as small as 500 microns and at least one air drawer 205 configured and arranged to draw ambient air towards the air filtration unit 204 and through the at least one filter 206. It should be noted that, although FIG. 2 depicts air drawer 205 as located sequentially before the at least one filter 206 in the air filtration unit 204, this air drawer 205 may be located sequentially after the at least one filter 206.

The at least one filter 206 capable of filtering out airborne particulate matter at least as small as 500 microns (roughly larger than the diameter of a human hair) is generally capable of filtering out smaller airborne matter, such as dust, soot, pollen, smoke, and microbes. In another aspect, the air filtration unit 204, either through a single filter 206 or a combination of filters, is capable of filtering out chemical and biological agents, microbes, allergens, radioactive contaminants (such as alpha, beta, and gamma particles), and the like.

Referring momentarily to FIG. 4, a single filter 400 is shown, with the arrows indicating the direction of airflow through the filter. The filter may comprise at least one of:

a high-efficiency particulate air (HEPA) filter;

an ultra low penetration air (ULPA) filter;

a super ultra low penetration air (SULPA) filter;

an activated carbon filter;

an electrostatic precipitator filter;

a charged media filter;

a gas phase filter;

a hybrid filter,

a charcoal filter;

a fiberglass filter;

a polyester filter;

a mechanical filter;

an electronic filter;

a ceramic filter;

a carbon filter;

a diatomaceous earth (DE) filter, and/or

a high efficiency gas adsorber (HEGA) filter;

to name but a few illustrative examples.

In one aspect, the at least one filter 206 is capable of removing particles of 0.3 microns or larger in diameter, such as a HEPA filter. HEPA filters are generally 99.99% efficient in removing particles of 0.3 microns or larger in diameter. In another aspect, the at least one filter 206 is capable of removing particles of 0.12 microns or larger in diameter, such as an ULPA or SULPA filter. ULPA filters are generally 99.999% efficient in removing particles of 0.12 microns or larger in diameter while SULPA filters are generally 99.9999% efficient in removing particles of 0.12 microns or larger in diameter.

By one optional approach, the at least one filter 206 may be pre-treated with an antimicrobial agent, such as hydrogen peroxide, enzymes, or quaternary ammonias, among others. By another approach, the at least one filter 206 may be periodically treated, as necessary, with an antimicrobial agent, such as with an antimicrobial spray. Use of a filter pretreated or periodically treated with an antimicrobial agent substantially reduces the ability of microbes to proliferate on the filter and, therefore, reduces the ability of microbes to contaminate the air filtration unit 204 and ambient air downstream of the filter. Filters treated with enzymes, such as lytic enzymes capable of degrading bacterial cell walls or viral envelopes, are commercially available, such as by Cambridge Filter Corporation (Gilbert, Ariz.).

By yet another optional approach, the at least one filter 206 may be scent-infused. Alternatively, the air filtration unit 204 may further comprise an aromatic scent releaser 208, as shown in FIG. 2, that is positioned to permit filtered air being expelled into the ambient air to be purposefully infused with a predetermined aromatic scent. Such an arrangement can provide for a pleasant aroma within a room or enclosed space and/or may mask any unpleasant odors in the ambient air.

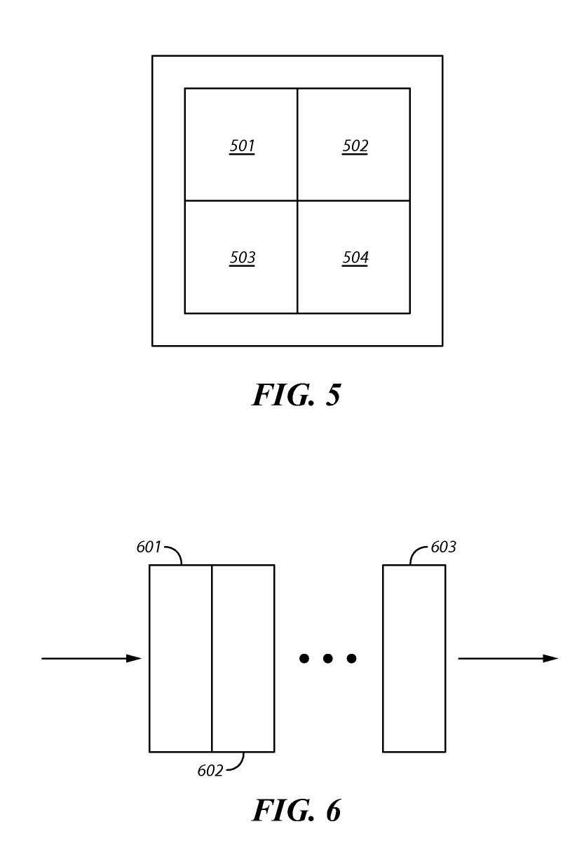

The at least one filter 206 of the robot 201 may also comprise multiple filters. The multiple filters may be arranged in a side-by-side configuration. Referring now to FIG. 5, multiple filters (501, 502, 503, and 504) are shown. In this illustration, four filters are arranged in a single layer in the general shape of a square within the filtration unit. It should be noted that the multiple filters may comprise any number of filters that are arranged in a single layer in a side-by-side configuration, wherein the side-by-side configuration may occur in a horizontal and/or vertical direction.

It is also possible for the multiple filters to be arranged in a stacked configuration. Referring now to FIG. 6, multiple filters (601, 602, and 603) are shown. In this illustration, the filters are stacked, with the face of each filter in mating contact with the face of an adjacent filter. However, it should be noted that the filters may also be arranged in a stacked configuration with an offset or spacing between each filter. The stacked configuration provides multiple layers of filters for the air to pass through. The stacked configuration may also be used in conjunction with the side-by-side configuration, as discussed above.

By one optional approach, the multiple filters arranged in the stacked configuration may be a same type of filter. Conversely, the multiple filters arranged in the stacked configuration may be different types of filters. For example, the filter 601 may be of a different type than the other filters 602 and 603 in the stack. The filters may be, for example, a combination of any of the varieties identified above. The use of different types of stacked filters may provide for more comprehensive filtration, wherein each filter works to filter out specific types and/or sizes of airborne particulate matter. For example, use of a filter capable of removing particles of 0.3 microns or larger in diameter and an activated carbon filter is effective in filtering out both bacterial and chemical contaminants.

Further, the at least one filter may be configured and arranged in the air filtration unit to facilitate a user accessing the at least one filter while preventing exposure to contaminants on the at least one filter. Accessing the at least one filter may further comprise removing the at least one filter at a filter access point on the exterior of the robot and replacing the at least one filter with at least one replacement filter. In another aspect, the at least one filter may be configured and arranged in the air filtration unit to facilitate a user accessing the at least one filter during operation of the filtration unit while preventing exposure to contaminants on the at least one filter.

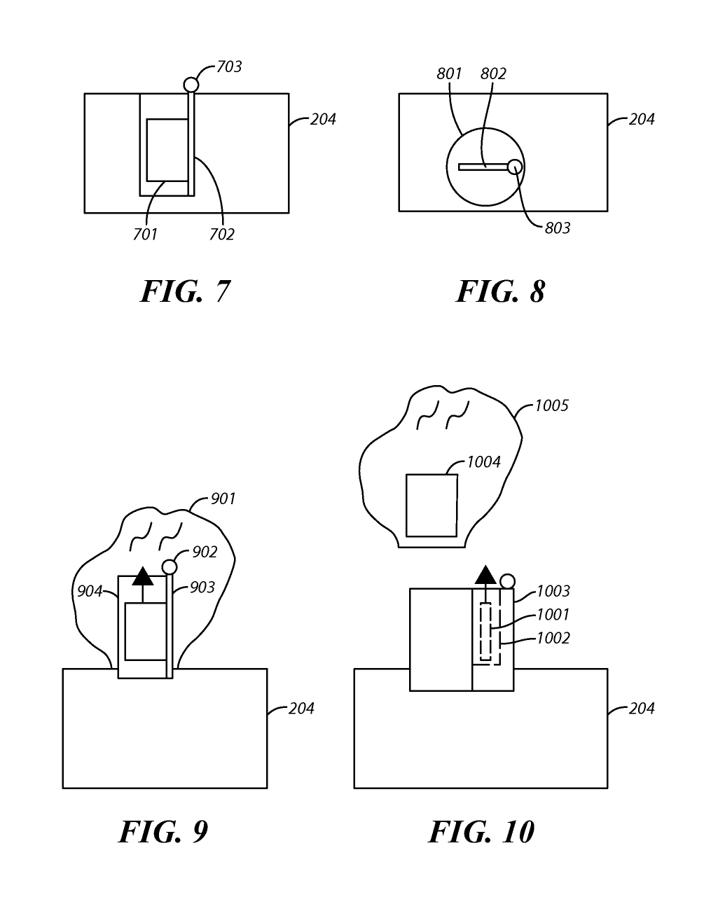

Many options are available for configuring and arranging the at least one filter to be accessed while preventing exposure to contaminants. An illustrative example of one such configuration is shown in FIG. 7. This illustration is similar to bag-in/bag-out systems known in the art. In this illustration, the at least one filter 701 is positioned in an air filtration unit 204. The at least one filter 701 is situated on a sliding platform 702. A knob 703 is connected to the sliding platform 702.

As shown in FIG. 8, when the at least one filter needs to be accessed, a user attaches a bag (shown in FIG. 9) to a bagging ring 801 around a filter access point 802. The bag and bagging ring 801 form a seal that prevents the escape of contaminants. In one aspect, where the air filtration unit 204 comprises more than one filter 206, each filter 206 may have a filter access point and sliding platform. In another aspect, more than one filter 206 may share a filter access point and be affixed to the same sliding platform.

As shown in FIG. 9, once the bag 901 has been secured to the bagging ring (shown in FIG. 8), the user may pull a knob 902 connected to the sliding platform 903. Pulling the knob 902 will move the sliding platform 903 toward the user, thereby moving the at least one filter 904 through the filter access point (as shown in FIG. 8) toward the user. The at least one filter 904 is moved toward the user when the sliding platform 903 is pulled into an extended position. The bag 901 serves as a barrier between the user and the at least one filter 904 at all times. The bag may be sealed by any means known in the art. Once the user removes the at least one filter 904 in the bag 901, the user may replace the old filter with a new replacement filter.

In another aspect, the replacement filter may also be prepositioned within the air filtration unit. By one optional approach, the configuration of FIG. 9 may further comprise a prepositioned replacement filter. As shown in FIG. 10, a replacement filter 1001 may be stored in a filter cavity 1002 that is attached to the sliding platform 1003. Therefore, after a user removes the at least one filter 1004 in the bag 1005. The replacement filter 1001 may then be removed from the filter cavity 1002 and inserted in the place of the original filter 1004.

As mentioned above, and referring again to FIG. 2, the air filtration unit 204 also comprises at least one air drawer 205 to draw ambient air toward the air filtration unit 204 and through the at least one filter 206. The at least one air drawer 205 is generally capable of drawing the ambient air through the filter and expelling the filtered air back into the ambient air, although the expelling action may optionally be provided by at least one air circulator 207, which works alone or in conjunction with the air drawer 205 to expel the filtered air. The air drawer 205 and/or the air circulator 207 serve to expel filtered air into the ambient air, thus providing a generally clean and sterile supply of air into an enclosed space.

The at least one air drawer 205 may comprise multiple air drawers. The multiple air drawers may be used concurrently or may be used independently. The additional air drawers may operate as a back-up to a primary air drawer, or the air drawers may operate to supplement the other air drawers to thereby increase or decrease the filtration of air as needed. In one aspect, each air drawer may be configured and arranged to draw air through at least one filter 206.

The multiple air drawers may be the same type of air drawer, or may be different types of air drawers. In this aspect, the drawing power of each air drawer can be optimized to provide adequate drawing power for a particular type of filter or combinations of filters. For example, filtration through a SULPA filter generally requires greater drawing power than for a HEPA filter.

With momentary reference to FIG. 3, the robot 201 may further comprise at least one germicidal energy source 305 configured and arranged to emit germicidal energy within the air filtration unit 204. By one optional approach, the at least one germicidal energy source 305 may be configured and arranged to emit germicidal energy within the filtration unit 204, preferably such that the ambient air entering the filtration unit 204 is treated by the germicidal energy source prior to the ambient air being filtered through the at least one filter. Such an arrangement provides an additional level of sterilization and may serve to reduce the amount of particulate matter passing through the at least one filter, thus extending the life of the filter(s). Such an arrangement also reduces the ability of microbes to proliferate on the filter and, therefore, reduces the ability of microbes to contaminate the air filtration unit 204 and ambient air downstream of the filter.

In one aspect, the germicidal energy source 305 may be a heat-generating device capable of producing temperatures of at least about 80° C., preferably in the range of about 80 to about 220° C., comprising a heated pipe through which ambient air is drawn by an air drawer 205 before the ambient air passes through a filter 206. The heat-generating device may also be in the form of heated plates through which ambient air is drawn by an air drawer 205 before passing through a filter 206.

In another aspect, the germicidal energy source 305 may comprise a 254 nm UV lamp, although for most purposes the 254 nm UV lamp should be configured and arranged to not emit UV radiation directly on the filter 206 in order to prevent damaging the filter.

In yet another aspect, the germicidal energy source 305 may comprise an RFEF apparatus where electric field strengths of up to 20 kilovolts per centimeter and frequencies in the range of 15 to 70 kilohertz are directed within the air filtration unit 204, such as within a pipe or tube through which the ambient air is drawn before being drawn through the at least one filter 204.

In yet another aspect, the germicidal energy source 305 may comprise an electrostatic apparatus such as an electrostatic precipitator as is well known in the art, where airborne particulate matter is drawn into the air filtration unit 204 by an air drawer 205 and subjected to an electric field, whereby the particulate matter becomes charged and passes through a collection device containing both positive and negative electrodes where the charged particulate matter is deposited on the electrodes, before the ambient air is drawn through the at least one filter 204.

Further, the germicidal energy source 305 may comprise any combination of one or more 254 nm UV lamps, RFEF apparatuses, electrostatic apparatuses, or heat producing devices capable of producing temperatures of at least about 80° C.

Referring again to FIG. 2, the robot 201 may further comprise at least one power source 209. The robot 201 may be powered in any of a variety of ways, including fuel powered, such as by hythane, biomethane, natural gas, bioethanol, and petrol, among others, or a combination thereof. The robot 201 may also be electrically powered. The electrical power may be provided, for example, by direct current sources and/or alternating current sources including but not limited to electrical power sourced by a generator, a battery, a solar cell, a thermoelectric source, and so forth.

In addition, the robot 201 may be powered, at least in part, by a human-powered crank. The human-powered crank allows a person to manually turn the crank to thereby operate the air drawer, and may comprise any of a variety of configurations, such as, for example, a handle or a pedal. Such a crank may be manipulable by hand, foot, or otherwise as may be appropriate in a given setting.

In one aspect, the at least one power source 209 may be at least one rechargeable battery. At full charge, the at least one rechargeable battery should be capable of providing sufficient power to run the robot 201 and provide sufficient power to any additional components, such as, but not limited to, the air filtration unit 204, for at least about sixty minutes, preferably at least about 90 minutes (though shorter amounts of time may be adequate for some application settings). In this aspect, the robot 201 further comprises a power connector 210 that is configured and arranged to electrically couple the robot 201 to an external docking station in order to recharge the at least one battery. Generally, the external docking station is placed in a location that will not interfere with the movement of inhabitants of the building or enclosed space and that is easily accessed by the robot 201. By one approach, the robot 201 automatically returns to the docking station to recharge the at least one battery when the remaining battery charge has decreased to a predetermined level.

In another aspect, the robot 201 utilizes solar energy as a source of power, where the power source 209 comprises, at least in part, a photovoltaic array or photoelectric cells. In this respect, the robot 201 is powered by natural light, such as by sunlight entering through a window, or artificial light, such as by light emitted from light fixtures in the enclosed space.

The power source 209 may also comprise, if desired, a power cord that operably connects the robot 201 to a docking station. Generally, the power cord is dimensioned such that the robot 201 is able to freely maneuver within the entire expanse of the enclosed space. Generally, the power cord is reversibly attached to the robot 201 and the docking station so that the power cord can easily be removed in case of entanglement with an object or objects in the enclosed space.

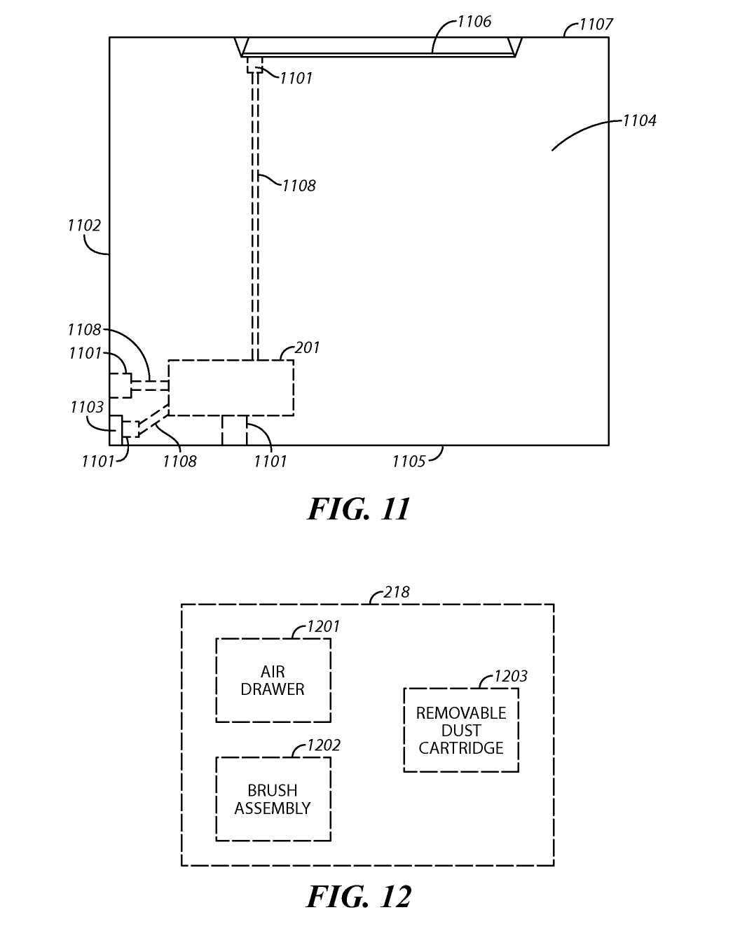

In yet another aspect, as shown in FIG. 11, the power source 209 may comprise one or more commutator interfaces 1101 that permit contact with an external electrically conducting device. In one aspect, the external electrically conducting device may be a wall 1102, including any parts of the wall such as a baseboard 1103, in an enclosed space 1104. In another aspect, the external electrically conducting device may be an electrically conducting floor 1105, such as where electrical wires are embedded in a flooring material or such as where the grout between floor tiles contains an electricity-conducting material. In this aspect, the commutator interface may comprise spring-based metal disks or metal shoes that contact the external electrically conducting device. In these aspects, the commutator interface 1101 may further comprise an elongated structure 1108, such as a rod, pole, or the like, that extends the commutator interface 1101 laterally and/or elevationally from the robot 201 to contact an external electrically conducting device.

In an alternative aspect, the electricity conducting device may be electrified overhead line(s) 1106 connected to the ceiling 1107 or walls 1102 of the enclosed space 1104. In this aspect, the commutator interface 1101 comprises an elongated structure 1108 that elevates the commutator interface 1101 from the robot 201 to contact the electrified overhead line(s) 1106.

By one approach, the power source 209 may comprise multiple power sources. The multiple power sources may be used concurrently or independently to provide alternate or redundant power supplies. Further, it may be desired to have different power sources for different components of the robot 201. For example, the motive capability may be powered by a first rechargeable battery while the germicidal energy source is powered by a second rechargeable battery. Having multiple power sources extends the operating life and allows the robot 201 to be used for extended periods of time.

If desired, the robot 201 may further comprise at least one environmental sampling device 211. The at least one environmental sampling device 211 is configured and arranged to take samples using at least one of the following sampling methods: swab sampling, sponge sampling, direct surface sampling, or air sampling. The at least one environmental sampling device allows for the analysis and detection of contamination by sampling various locations about an enclosed space. Locations of sampling may be random or predetermined.

In another aspect, the at least one environmental sampling device also allows for the evaluation of the efficacy of the sterilization by the robot 201. Locations of sampling may be predetermined in order to evaluate whether the sterilization previously completed by the robot 201 in that location were effective in reducing or eliminating contamination.

Generally, swab sampling, sponge sampling, and direct surface sampling should be capable of detecting indicators of contaminated air or surfaces, such as aerobic plate count, psychotrophic plate count, Enterobacteriaceae, coliform, yeast, mold, and adenosine triphosphate (ATP). In some cases it may be useful or desirable that the samples collected by the environmental sampling device 211 be analyzed by an automated external work station, such as by the docking station. Some illustrative examples in this regard would include, but are not limited to, an automated external work station capable of detecting microorganisms in a sample by polymerase chain reaction (PCR) or ATP bioluminescence. In another aspect, the environmental sampling device 211 collects samples and stores the samples until a user can retrieve the samples and transfer them to an external laboratory facility for appropriate analysis.

In another aspect, the environmental sampling device 211 may be the same as the air filtration unit 204. For example, air samples may be taken by drawing ambient air by the air drawer 205. These air samples may be analyzed by numerous different sensors, such as, but not limited to, the sensors discussed below.

Referring momentarily to FIG. 1, the method may further comprise using the robot 102 to detect 103 a predetermined external condition. Detection 103 of a predetermined external condition results in one or more predetermined responses 104 by the robot. Referring again to FIG. 2, such predetermined external conditions may be detected using at least one sensor 212. The term “sensor” is interchangeable with the term “detector.” In one aspect, the at least one sensor 212 is located on the exterior of the robot 201, such that the at least one sensor 212 contacts ambient air. In this aspect, the at least one sensor 212 may be located on the surface of the robot 201 or may be laterally or elevationally extended from the surface of the robot 201, such as by a pole, shaft, rod, or the like. In another aspect, the at least one sensor 212 is located on the interior of the robot 201, such as within the air filtration unit 204. In yet another aspect, the at least one sensor is remote to the robot 201, such as located on a docking station.

The at least one sensor 212 generates a signal that is transmitted to a control system 213, which directs a predetermined response. It should be noted that the control system 213 may be part of the sensor 212 and is not necessarily a separate device. Numerous different types of sensors and predetermined responses are envisioned, many of which are explained below.

In one aspect of the invention, the sensor 212 is capable of detecting the presence of at least one of the group consisting of a human, animal, and visible light, such as from an incandescent light bulb. For example, if a person or animal, such as a dog or cat, enters a room in which the robot 201 is operating, the germicidal energy source 202 could pose a danger to the person or animal, particularly if the germicidal energy source 202 is one or more 254 nm UV lamps, which can cause damage to the skin and eyes. Therefore, for at least some application settings, it may be useful for the robot 201 be able to detect the presence of a human or animal and respond in a way that reduces any inconvenience that might be posed to the human or animal.

The sensor capable of detecting the presence of a human, animal, or visible light may be an infrared sensor, a motion detector, or a combination of both. The infrared sensor detects changes in infrared heat in the enclosed space, thus detecting the heat emitted by a person or animal or detects the presence of a person by detecting the heat given off by a light fixture where, for example, a person enters a room and turns on a light. Alternatively or additionally, the sensor may be a motion detector. Motion detectors typically comprise infrared sensors, ultrasonic sensors (such as where ultrasonic pulses are emitted and the motion detector measures the reflection of a moving object), or microwave sensors (such as where microwaves are bounced of an object).

Generally, detection of a human, animal, and/or visible light results in the generation of a signal by the sensor 212 that is transferred to control system 213. The control system 213 then generates a signal that results in one or more predetermined responses. By one approach the control system 213 generates a signal that is transmitted to an audio output device 214 that generates an audible warning to alert the human that a robot is moving in the enclosed space. The warning also serves to alert the human of the robot's presence in order to prevent the human from being exposed to the at least one germicidal energy source 202. In this aspect, an audio output device 214 generates an audible warning, such as a voice command, beep, siren, musical note, chime, alarm bell, or the like. The audible warning may also include a sound imperceptible to human hearing but that would be unpleasant to an animal such that the animal would be encouraged to exit the enclosed space.

In another aspect, detection of a human, animal, or visible light results in the generation of a signal by the control system 213 that is transmitted to the germicidal energy source 202 to trigger the at least one germicidal energy source 202 to turn off. This aspect is particularly useful when the germicidal energy source 202 is a 254 nm UV lamp that could potentially cause damage to the skin and eyes of a human or animal.

In yet another aspect, detection of a human, animal, and/or visible light results in the generation of a signal by the control system 213 that is transmitted to the motive capability 203 to trigger the robot 201 to move to couple to a docking station or to trigger the robot 201 to pause the robot's movement for a predetermined period of time. Triggering the robot 201 to dock at the docking station or to pause allows a human or animal to maneuver safely in the enclosed space without the robot 201 running into the human or animal or causing the human or animal to otherwise be inconvenienced or confused.

The sensor 212 may comprise a debris detecting sensor capable of detecting debris on a surface, such as a floor surface. Detection of debris results in generating a signal that is transmitted to control system 213. Control system 213 then generates a signal that is transmitted to the motive capability 203 which results, for example, in pausing the robot's movement for at least a predetermined length of time, such as about five seconds. This allows the robot 201 to concentrate cleaning and/or sterilization activities in a location having debris, which is also more likely to have a greater concentration of contaminants.

The sensor 212 may comprise a beacon sensor. The beacon sensor monitors to detect a signal from at least one beacon 703 (as depicted in FIG. 7). The at least one beacon 703 may be located in various locations within an enclosed space. Detection of a signal from a beacon 703 by a beacon sensor causes the beacon sensor to generate a signal that is transmitted to a control system 213. The control system 213 then generates a signal that is transmitted to the motive capability 203 which causes the robot 201 to focus its sterilization activity in an area that is proximal to the at least one beacon. For example, it may be desired that the robot 201 focuses its sterilization activity on heavy traffic areas, such as hallways or doorways. In this aspect, at least one beacon is located in the area desired to be cleaned and/or sterilized. In an alternative aspect, the at least one beacon may be used to cause the robot 201 to focus its sterilization activity in an area that is remote from the at least one beacon. In this aspect, detection of a signal from a beacon 703 by a beacon sensor causes the control system 213 to generate a signal that is transmitted to the motive capability 203 which causes the robot 201 to change direction and move away from the beacon. For example, it may be desired that the robot stay away from high traffic areas at times when inhabitants are expected to be present, such as during working hours.

The sensor 212 may comprise at least one environmental sensor. The environmental sensor is capable, for example, of detecting at least one of the following environmental conditions: temperature, humidity, barometric pressure, smoke, radon, and ionizing radiation (such as by alpha, beta, or gamma particles). Generally, detection of an environmental condition above a predetermined level, such as a level above which the condition poses a danger to an inhabitant of the enclosed space, results in the generation of an audible warning as described above.

The sensor 212 may also comprise a chemical agent sensor that is capable of detecting at least one agent selected from the group consisting of the following categories of chemical agents: biotoxin, blister agent/vesicant, blood agent, caustic agent, choking/lung/pulmonary agent, incapacitating agent, long-acting anticoagulant, metal agent, nerve agent, organic solvent, riot control agent/tear gas, toxic alcohol, and vomiting agent.

An illustrative but non-exhaustive listing of exemplary chemical agents for each category of chemical agents includes, but are not limited to, the following:

biotoxins, which generally includes poisons that originate from plants or animals, such as abrin, brevetoxin, colchicine, digitalis, nicotine, ricin, saxitoxin, strychnine, tetrodotoxin, and trichothecene;

blister agents/vesicants, which generally includes chemicals that may severely blister the eyes, respiratory tract, or skin upon contact, such as mustard agents (distilled mustard (HD), mustard gas (H) (sulfur mustard), mustard/lewisite (HL), mustard/T, nitrogen mustard (HN-1, HN-2, HN-3), sesqui mustard), Lewisites/chloroarsine agents (Lewisite (L, L-1, L-2, L-3), mustard/lewisite (HL)), and phosgene oxime (CX);

blood agents, which generally includes poisons that affect the body by absorption into the blood, such as arsine (SA), carbon monoxide, cyanide, cyanogens chloride (CK), hydrogen cyanide (AC), potassium cyanide (KCN), sodium cyanide (NaCN), and sodium monofluoroacetate (compound 1080);

caustic agents, which generally includes chemicals that burn or corrode the skin, eyes, and mucus membranes upon contact, such as hydrofluoric acid;

choking/lung/pulmonary agents, which generally includes chemicals that cause severe irritation or swelling of the respiratory tract (nose, throat, and lungs), such as ammonia, bromine (CA), chlorine, hydrogen chloride, methyl bromide, methyl isocyanate, osmium tetroxide, phosgene (including diphosgene (DP) and phosgene (CG)), phosphine, elemental phosphorus, and sulfuryl fluoride;

incapacitating agents, which generally includes drugs that cause people to be unable to think clearly or that cause unconsciousness or an altered state of consciousness, such as BZ or fentanyls or other opioids;

long-acting anticoagulants, which generally includes poisons that prevent blood from clotting properly, causing uncontrolled bleeding, such as super warfarin;

metal agents, which generally includes agents that consist of metallic poisons, such as arsenic, barium, mercury, and thallium;

nerve agents, which generally includes highly poisonous chemicals that prevent the nervous system from working properly, such as G agents (sarin (GB), soman (GD), tabun (GA)) and V agents, such as S-(Diethylamino)ethyl O-ethyl ethylphosphonothioate (VE), O,O-Diethyl-S-[2-(diethylamino)ethyl]phosphorothioate (VG), Phosphonothioic acid, methyl-, S-(2-(diethylamino)ethyl) O-ethyl ester (VM), or O-ethyl-S-[2(diisopropylamino)ethyl]methylphosphonothioate (VX);

organic solvents, which generally includes agents that damage living tissue by dissolving fats and oils, such as benzene;

riot control agents/tear gas, which generally includes highly irritating agents normally used for crowd control or by individuals for personal protection, such as bromobenzylcyanide (CA), chloroacetophenone (CN), chlorobenzylidenemalonitrile (CS), chloropicrin (PS), and dibenzoxazepine (CR);

toxic alcohols, which generally includes poisonous alcohols that damage the heart, kidneys, and nervous system, such as ethylene glycol; and

vomiting agents, which generally includes chemicals that cause nausea and vomiting, such as adamsite (DM).

Generally, detection by the sensor 212 of a given chemical agent above a predetermined level, such as a level above which the chemical agent poses a danger to an inhabitant of the enclosed space, results in the generation of a signal by the sensor 212 that is transmitted to the control system 213. The control system 213 then generates a signal that is transmitted to an audio output device 214 that generates an audible warning as described above.

The sensor 202 may comprise an allergen sensor that is capable of detecting at least one of the following airborne allergens: ragweed, dust, dust mites, pollen, pet dander, and mold spores. Generally, detection by the sensor 212 of an allergen above a predetermined level, such as a level above which the allergen may adversely affect the health of or pose a danger to an allergic inhabitant of the enclosed space, results in the generation of an audible warning as described above.

The robot 201 may further comprise a data transmitter 215. The data transmitter 215 may be a data transmitter cord operably connected to an external docking station or a wireless transmitter. In one aspect, a signal generated by any of the sensors described above may be transmitted via a wireless transmitter or a data transmitter to an external docking station, where the generated signal is stored and/or further relayed as data by the docking station. In another aspect, a signal generated by any of the sensors described above may be transmitted via a wireless transmitter to a remote computer where the generated signal is stored as data by the computer. Thus, in both of these aspects, the conditions in the enclosed space may be monitored on a continuous basis or on an as-needed basis.

The robot 201 may further comprise a global positioning satellite (GPS) receiver 216. By one approach, the GPS receiver allows for the movements of the robot 201 to be documented and tracked. The data may then be transmitted via a data transmitter 215 to an external docking station or to a remote computer where the data is stored. By this approach, it is possible to determine whether the movements of the robot 201 have achieved the desired coverage, such as full coverage, of the enclosed space. By another approach, the GPS receiver allows a user to control the movements of the robot 201 and can define the area, such as by selecting a particular grid, within which the apparatus must stay, such as limiting the movements of the robot 201 to particular rooms within a building. In some cases, the application setting may partially or fully block such GPS signals; in such a case, other location-based tracking systems of choice can be similarly employed to achieve similar purposes. In other cases, the application setting may require the use of relays and boosters to transmit the GPS signal.

In some cases it may be useful or desirable that samples gathered from the environmental sampling device be used to identify locations with unacceptable levels of contamination or to identify locations that frequently exhibit unacceptable levels of contamination. In this respect, the robot 201 may be controlled or programmed using the GPS receiver 216 to focus the robot 201's movements to certain predetermined locations within the enclosed space. For example, it may be determined that certain locations within a building warrant particularly strict microbial detection limits, such as a particular wing in a hospital where immunocompromised patients are housed or in a daycare facility where young children play.

The robot 201 may further comprise a radio frequency identification (RFID) tag (or transponder) 217. The RFID tag 217 may be a passive tag (requiring no internal power) or may be an active tag (requiring a power source), as are well known in the art. The RFID tag 217 generally comprises a transponder that transmits data to provide identification and/or location information.

The robot 201 may further comprise a vacuum device 218 as known in the art. The interested reader is directed to U.S. Pat. No. 6,683,201, which is incorporated herein by reference. As shown in FIG. 12, the vacuum device 218 generally comprises an air drawer 1201, brush assembly 1202, and removable dust cartridge 1203. The removable dust cartridge 1203 may be a filter. In one aspect, the air drawer 1201 comprises the air drawer 205 that comprises a part of the air filtration unit 204. In another aspect, this air drawer 801 is distinct and separate from the air drawer 205 in the air filtration unit 204.

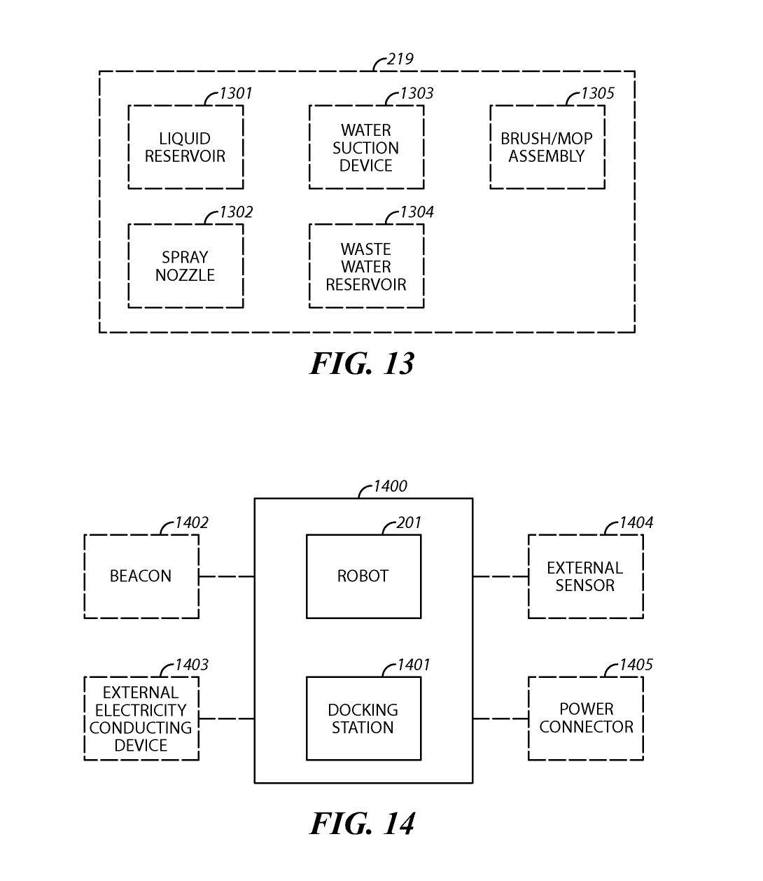

The robot 201 may further comprise a floor washing device 218 as known in the art. As shown in FIG. 13, the floor washing device 219 generally comprises a liquid reservoir 1301, a spray nozzle 1302, a water suction device 1303, and a waste water reservoir 1304. The floor washing device 219 may optionally further comprise a brush or mop assembly 1305. The liquid reservoir 1301 contains the liquid used for cleaning floor surface, such as floor cleaners, bleach, water, detergents, or germicidal liquids. The liquid is sprayed onto the floor surface by the spray nozzle 1302. Optionally, the floor washing device may include a brush or mop assembly 1305 that brushes or mops the floor. The water suction device 1303 removes the liquid from the floor surface and stores the liquid in the waste water reservoir 1304.

The robot 201 may further comprise an activator 220. The activator 220 may comprise at least one of an automated timer or a remotely-operated activator. A remotely-operated activator enables a user to turn on the robot from a remote location. Alternatively, an automated timer enables a user to turn on the robot's sterilization action on a pre-scheduled or predetermined basis. Both types of activators allow a user to activate the robot 201 to sterilize enclosed spaces without requiring the user to be physically present to activate the robot. It should also be noted that the activator may activate all of the components of the robot 201 or may activate only particular component(s) of the robot 201. For example, it may desired to activate the air filtration unit 204 but not the germicidal energy source 202 or the sensor capable of detecting the presence of humans, animals, or visible light when persons or animals are expected to be present.

The robot 201 may further comprise a waterproof housing 221 that encapsulates at least the electronic components of the apparatus. As is known in the art, such a waterproof housing 221 may comprise known waterproof materials and/or waterproof gaskets or may be achieved by using known manufacturing techniques such as ultrasonic welding or the like.

The robot 201 may further comprise a padded housing 222 that covers at least the corners and any protruding surfaces of the robot 201. In one aspect, the padded housing 222 comprises foam padding or foam rubber padding, as are readily known in the art,

Referring now to FIG. 14, the robot 201 may be provided in a robot system 1400. The robot system 1400 comprises a robot 201 with any of the components discussed above and a docking station 1401. The robot system 1400 may further comprise a beacon 1402 as described above in the discussion of beacon sensors. The robot system 1400 may further comprise an external electricity conducting device 1403 as described above. The robot system 1400 may also comprise at least one external sensor 1404. The external sensor 1404 may be any one of the sensors 212 discussed above or a combination thereof. The robot system 1400 may further comprise a power connector 1405 configured and arranged to electrically couple the robot to the docking station as discussed above.

Those skilled in the art will recognize and appreciate that these teachings provide for a highly flexible yet powerfully effective apparatus and method for neutralizing microbes that pose potential risks to human and/or animal inhabitants. Importantly, these teachings provide a relatively cost effective approach to which microbes cannot develop resistance over time. Moreover, these teachings can readily be applied in a variety of application settings. Additionally, a variety of activities, such as general cleaning, environment monitoring, among others, can be accommodated if desired in a shared platform.

Those skilled in the art will recognize that a wide variety of modifications, alterations, and combinations can be made with respect to the above described embodiments without departing from the spirit and scope of the invention, and that such modifications, alterations, and combinations are to be viewed as being within the ambit of the inventive concept.

As but one illustrative example, the robot may operate in a submerged manner. In such application settings, the filter may comprise a diatomaceous earth (DE) filter. For example the robot may be used in water treatment facilities to control or improve water quality. In another aspect, it may be desired to control water quality in water storage facilities or pipes carrying water.

As another example in this regard, these teachings may be scaled with specific application settings in mind. In one aspect, the robot may be relatively small, such as for use in ductwork or pipes. Or if desired, the robot may be quite large, such as the form factor of a street sweeper. By this approach, substantially larger volumes of air may be filtered and/or sterilized.

As yet another example in this regard, as the scale of the robot changes, different modes of piloting may be provided, such as autonomous piloting, real time remote piloting, or piloting by a passenger (such as a person piloting a street sweeper).

As yet another example in this regard, depending on the application setting, it would also be possible to use a higher energy source for the germicidal energy source, including X-rays, microwaves, and electron beam technologies.

We claim:

1. An apparatus comprising:
a robot configured and arranged to move over a surface;
at least one germicidal energy source carried by the robot;
at least one motive capability to facilitate movement of the robot over the surface.

2. The apparatus of claim 1 wherein the germicidal energy source carried by the robot is selected from the group consisting of:
ultraviolet (UV) lamps;
radiofrequency electric field (RFEF) apparatuses;
electrostatic apparatuses;
heat generating devices capable of producing heat at a temperature of at least 80° C.; or
a combination thereof.

3. The apparatus of claim 2 wherein the germicidal energy source is configured and arranged to emit germicidal energy outwardly from the robot.

4. The apparatus of claim 3 wherein the germicidal energy source configured and arranged to emit germicidal energy outwardly from the robot comprises at least one 254 nanometer UV lamp.

5. The apparatus of claim 4 wherein the at least one 254 nanometer UV lamp comprises multiple UV lamps.

6. The apparatus of claim 5 wherein the multiple UV lamps are aimed in a plurality of outward directions.

7. The apparatus of claim 5 wherein at least one of the multiple UV lamps is configured and arranged to emit UV waves outwardly towards a floor surface and at least one of the multiple UV lamps is configured and arranged to emit UV waves outwardly into the ambient air.

8. The apparatus of claim 1 further comprising an air filtration unit that is carried by the robot, wherein the air filtration unit comprises:
at least one filter capable of filtering out airborne particulate matter at least as small as 500 microns, wherein the at least one filter comprises at least one of:
high-efficiency particulate air (HEPA) filter;
ultra low penetration air (ULPA) filter;
super ultra low penetration air (SULPA) filter;
activated carbon filter;
electrostatic precipitator filter;
charged media filter;
gas phase filter;
hybrid filter,
charcoal filter;
fiberglass filter;
polyester filter;
mechanical filter;
electronic filter;
ceramic filter;
carbon filter;
high efficiency gas adsorber (HEGA) filter; and
at least one air drawer capable of drawing ambient air toward the self-propelled sterilization robot and through the at least one filter.

9. The apparatus of claim 8 wherein the air filtration unit further comprises at least one air circulator capable of circulating filtered air into the ambient air.

10. The apparatus of claim 8 wherein the air filtration unit further comprises an aromatic scent releaser positioned to permit air being expelled into the ambient air to be purposefully infused with a predetermined aromatic scent.

11. The apparatus of claim 8 wherein the at least one filter is capable of filtering out airborne particulate matter at least as small as 0.3 microns.

12. The apparatus of claim 8 wherein the at least one filter comprises multiple filters.

13. The apparatus of claim 12 wherein the multiple filters comprises at least one filter capable of filtering out airborne particulate matter of at least as small as 0.3 microns and at least one activated carbon filter.

14. The apparatus of claim 12 wherein the multiple filters are arranged in a side-by-side configuration.

15. The apparatus of claim 14 wherein the multiple filters arranged in the side-by-side configuration are a same type of filter.

16. The apparatus of claim 14 wherein the multiple filters arranged in the side-by-side configuration are different types of filters.

17. The apparatus of claim 12 wherein the multiple filters are arranged in a stacked configuration.

18. The apparatus of claim 17 wherein the multiple filters arranged in the stacked configuration are a same type of filter.

19. The apparatus of claim 17 wherein the multiple filters arranged in the stacked configuration are different types of filters.

20. The apparatus of claim 8 wherein the at least one air drawer comprises multiple air drawers.

21. The apparatus of claim 1 further comprising a control system.

22. The apparatus of claim 1 further comprising a vacuum device.

23. The apparatus of claim 1 further comprising a floor washing device.

24. The apparatus of claim 1 further comprising a sensor capable of detecting the presence of at least one of the group consisting of:
a human;
an animal;
visible light.

25. The apparatus of claim 24 wherein detection of a human, an animal, and/or visible light triggers the robot's movement to couple to a docking station.

26. The apparatus of claim 25 wherein detection of a human, an animal, and/or visible light generates an audible warning.

27. The apparatus of claim 25 wherein detection of a human, an animal, and/or visible light triggers the germicidal energy source to turn off.

28. The apparatus of claim 25 wherein the sensor comprises at least one of:
an infrared sensor;
a motion sensor.

29. The apparatus of claim 1 further comprising at least one germicidal energy source configured and arranged to emit germicidal energy inwardly toward the filtration unit.

30. The apparatus of claim 1 further comprising a power source selected from the group consisting of:
at least one rechargeable battery;
a power cord that operably connects to a docking station;
at least one commutator interface that permits contact to an external power supply.

31. The apparatus of claim 30 further comprising a power connector configured and arranged to electrically couple to a docking station to recharge the battery.

32. The apparatus of claim 1 further comprising a data transmitter.

33. The apparatus of claim 32 wherein the data transmitter is a wireless transmitter.

34. The apparatus of claim 32 wherein the data transmitter is a data transmitter cord connected to a docking station.

35. The apparatus of claim 1 further comprising an activator, wherein the activator comprises at least one of the following:
an automated timer activator;
a remotely-operated activator.

36. The apparatus of claim 1 further comprising a debris detecting sensor capable of detecting debris on a surface and generating a signal in response to detection of the debris, the signal triggering a pause in the robot's movement for at least a predetermined length of time.

37. The apparatus of claim 1 further comprising a global positioning system (GPS) receiver.

38. The apparatus of claim 1 further comprising a beacon sensor, wherein the beacon sensor monitors to detect a signal from a beacon, and generating a signal in response to detection of a beacon signal, the signal triggering the robot to particularly focus its sterilization activity in an area that is proximal to the beacon.

39. The apparatus of claim 1 further comprising a beacon sensor, wherein the beacon sensor monitors to detect a signal from a beacon, and generating a signal in response to detection of a beacon signal, the signal triggering the robot to particularly focus its sterilization activity in an area that is remote from the beacon.

40. The apparatus of claim 1 wherein the robot's sterilization activity movement is generally random in direction.

41. The apparatus of claim 1 further comprising at least one environment sensor, wherein the environmental sensor monitors to detect at least one of the following environmental conditions:
temperature;
humidity;
barometric pressure;
smoke;
radon;
ionizing radiation.

42. The apparatus of claim 41 wherein the environmental sensor generates a signal in response to detection of an environmental condition above a predetermined value.

43. The apparatus of claim 41 wherein detection of an environmental condition above a preset value generates an audible warning.

44. The apparatus of claim 41 wherein detection of an environmental condition above a preset value generates a signal that is transmitted to a docking station such that the generated signal is storable as data by the docking station.

45. The apparatus of claim 1 further comprising a chemical agent detection device, wherein the chemical agent detection device is capable of detecting at least one of the chemical agents selected from the group consisting of:
biotoxin;
blister agent/vesicant;
blood agent;
caustic agent;
choking/lung/pulmonary agent;
incapacitating agent;
long-acting anticoagulant;
metal;
nerve agent;
organic solvent;
riot control agent/tear gas;
toxic alcohol;
vomiting agent.

46. The apparatus of claim 45 wherein detection of a chemical agent above a predetermined value generates an audible warning.

47. The apparatus of claim 45 wherein detection of a chemical agent above a predetermined value generates a signal that is transmitted to a docking station such that the generated signal is storable as data by the docking station.

48. The apparatus of claim 1 further comprising a waterproof housing.

49. The apparatus of claim 1 further comprising an allergen sensor, wherein the allergen sensor detects at least one of the following allergens:
ragweed;
dust;
dust mites;
pollen;
pet dander; and
mold spores

50. The apparatus of claim 1 further comprising an environmental sampling device, wherein the environmental sampling device takes a sample using at least one of the following sampling methods:
swab sampling;
sponge sampling;
direct surface sampling;
air sampling.

51. The apparatus of claim 50 wherein the sampling method is capable of detecting at least one of the following indicators of contaminated air or surfaces:
aerobic plate count;
psychotrophic plate count;
Enterobacteriaceae;
coliform;
yeast;
mold;
adenosine triphosphate (ATP).

52. A robot system comprising:
a docking station; and
a robot, the robot comprising:
at least one germicidal energy source configured and arranged to emit germicidal energy outwardly from the robot;
at least one motive capability to facilitate the robot's movement on a surface;
a filtration unit for filtering the ambient air, the filtration unit comprising:
at least one filter capable of filtering out airborne particulate matter at least as small as 500 microns, wherein the at least one filter comprises at least one of:
high-efficiency particulate air (HEPA) filter;
ultra low penetration air (ULPA) filter;
super ultra low penetration air (SULPA) filter;
activated carbon filter;
electrostatic precipitator filter;
charged media filter;
gas phase filter;
hybrid filter,
charcoal filter;
fiberglass filter;
polyester filter;
mechanical filter;
electronic filter;
ceramic filter;
carbon filter;
high efficiency gas adsorber (HEGA) filter; and
at least one air drawer capable of drawing ambient air toward the self-propelled sterilization robot and through the at least one filter.

53. The robot system of claim 52 further comprising a power connector configured and arranged to electrically couple the robot to the docking station.

54. The robot system of claim 52 further comprising a beacon that is separate from the robot and wherein the robot further comprises a beacon sensor that is configured and arranged to influence control of the at least one motive capability.

55. The robot system of claim 52 further comprising an external electricity conducting device.

56. A method comprising providing a sterilization apparatus, the sterilization apparatus comprising:
a robot configured and arranged to move over a surface;
at least one germicidal energy source carried by the robot;
at least one motive capability to facilitate movement of the robot over the surface.

57. The method of claim 56 wherein the germicidal energy source carried by the robot is selected from the group consisting of:
ultraviolet (UV) lamps;
radiofrequency electric field (RFEF) apparatuses;
electrostatic apparatuses;
heat generating devices capable of producing heat at a temperature of at least 80° C.; or
a combination thereof.

58. The method of claim 57 wherein the germicidal energy source is configured and arranged to emit germicidal energy outwardly from the robot.

59. The method of claim 58 wherein the germicidal energy source configured and arranged to emit germicidal energy outwardly from the robot comprises at least one 254 nanometer UV lamp.

60. The method of claim 59 wherein the at least one 254 nanometer UV lamp comprises multiple UV lamps.

61. The method of claim 60 wherein the multiple UV lamps are aimed in a plurality of outward directions.

62. The method of claim 60 wherein at least one of the multiple UV lamps is configured and arranged to emit UV waves outwardly towards a floor surface and at least one of the multiple UV lamps is configured and arranged to emit UV waves outwardly into the ambient air.

63. The method of claim 56 wherein the robot further comprises an air filtration unit, wherein the air filtration unit comprises:
at least one filter capable of filtering out airborne particulate matter at least as small as 500 microns, wherein the at least one filter comprises at least one of:
high-efficiency particulate air (HEPA) filter;
ultra low penetration air (ULPA) filter;
super ultra low penetration air (SULPA) filter;
activated carbon filter;
electrostatic precipitator filter;
charged media filter;
gas phase filter;
hybrid filter,
charcoal filter;
fiberglass filter;
polyester filter;
mechanical filter;
electronic filter;
ceramic filter;
carbon filter;
high efficiency gas adsorber (HEGA) filter; and
at least one air drawer capable of drawing ambient air toward the self-propelled sterilization robot and through the at least one filter.

64. The method of claim 63 wherein the air filtration unit further comprises at least one air circulator capable of circulating filtered air into the ambient air.

65. The method of claim 63 wherein the air filtration unit further comprises an aromatic scent releaser positioned to permit air being expelled into the ambient air to be purposefully infused with a predetermined aromatic scent.

66. The method of claim 63 wherein the at least one filter is capable of filtering out airborne particulate matter at least as small as 0.3 microns.

67. The method of claim 63 wherein the at least one filter comprises multiple filters.

68. The method of claim 67 wherein the multiple filters comprises at least one filter capable of filtering out airborne particulate matter of at least as small as 0.3 microns and at least one activated carbon filter.

69. The method of claim 67 wherein the multiple filters are arranged in a side-by-side configuration.

70. The method of claim 69 wherein the multiple filters arranged in the side-by-side configuration are a same type of filter.

71. The method of claim 70 wherein the multiple filters arranged in the side-by-side configuration are different types of filters.

72. The method of claim 67 wherein the multiple filters are arranged in a stacked configuration.

73. The method of claim 72 wherein the multiple filters arranged in the stacked configuration are a same type of filter.

74. The method of claim 72 wherein the multiple filters arranged in the stacked configuration are different types of filters.

75. The method of claim 63 wherein the at least one air drawer comprises multiple air drawers.

76. The method of claim 64 wherein the at least one air circulator comprises multiple air circulators.

77. The method of claim 56 wherein the robot further comprises a vacuum device.

78. The method of claim 56 wherein the robot further comprise a floor washing device.

79. The method of claim 56 wherein the robot further comprises a sensor capable of detecting the presence of at least one of the group consisting of:
a human;
an animal;
visible light.

80. The method of claim 79 wherein detection of a human, an animal, and/or visible light triggers the robot's movement to couple to a docking station.

81. The method of claim 79 wherein detection of a human, an animal, and/or visible light generates an audible warning.

82. The method of claim 79 wherein detection of a human, an animal, and/or visible light triggers the germicidal energy source to turn off.

83. The method of claim 83 wherein the sensor comprises at least one of:
an infrared sensor;
a motion sensor.

84. The method of claim 56 wherein the robot further comprises at least one germicidal energy source configured and arranged to emit germicidal energy inwardly toward the filtration unit.

85. The method of claim 56 wherein the robot further comprises a power source selected from the group consisting of:
at least one rechargeable battery;
a power cord that operably connects to a docking station;
at least one commutator interface that permits contact to an external power supply.

86. The method of claim 85 wherein the robot further comprises a power connector configured and arranged to electrically couple to a docking station to recharge the battery.

87. The method of claim 56 wherein the robot further comprises a data transmitter.

88. The method of claim 87 wherein the data transmitter is a wireless transmitter.

89. The apparatus of claim 87 wherein the data transmitter is a data transmitter cord connected to a docking station.

90. The method of claim 56 wherein the robot further comprises an activator, wherein the activator comprises at least one of the following:
an automated timer activator;
a remotely-operated activator.

91. The method of claim 56 wherein the robot further comprises a debris detecting sensor capable of detecting debris on a surface and generating a signal in response to detection of the debris, the signal triggering a pause in the robot's movement for at least a predetermined length of time.

92. The method of claim 56 wherein the robot further comprises a global positioning system (GPS) receiver.

93. The method of claim 56 wherein the robot further comprises a beacon sensor, wherein the beacon sensor monitors to detect a signal from a beacon, and generating a signal in response to detection of a beacon signal, the signal triggering the robot to particularly focus its sterilization activity in an area that is proximal to the beacon.

94. The method of claim 56 wherein the robot further comprises a beacon sensor, wherein the beacon sensor monitors to detect a signal from a beacon, and generating a signal in response to detection of a beacon signal, the signal triggering the robot to particularly focus its sterilization activity in an area that is remote from the beacon.

95. The method of claim 56 wherein the robot's sterilization activity movement is generally random in direction.

96. The method of claim 56 wherein the robot further comprises at least one environment sensor, wherein the environmental sensor monitors to detect at least one of the following environmental conditions:
temperature;
humidity;
barometric pressure;
smoke;
radon;
ionizing radiation.

97. The method of claim 96 wherein the environmental sensor generates a signal in response to detection of an environmental condition above a predetermined value.

98. The method of claim 97 wherein detection of an environmental condition above a preset value generates an audible warning.

99. The method of 97 wherein detection of an environmental condition above a preset value generates a signal that is transmitted to a docking station such that the generated signal is storable as data by the docking station.

100. The method of claim 56 wherein the robot further comprises a chemical agent detection device, wherein the chemical agent detection device is capable of detecting at least one of the chemical agents selected from the group consisting of:
biotoxin;
blister agent/vesicant;
blood agent;
caustic agent;
choking/lung/pulmonary agent;
incapacitating agent;
long-acting anticoagulant;
metal;
nerve agent;
organic solvent;
riot control agent/tear gas;
toxic alcohol;
vomiting agent.

101. The method of claim 100 wherein detection of a chemical agent above a predetermined value generates an audible warning.

102. The method of claim 101 wherein detection of a chemical agent above a predetermined value generates a signal that is transmitted to a docking station such that the generated signal is storable as data by the docking station.

103. The method of claim 56 wherein the robot further comprises a waterproof housing.

104. The method of claim 56 wherein the robot further comprises an allergen sensor, wherein the allergen sensor detects at least one of the following allergens:
ragweed;
dust;
dust mites;
pollen;
pet dander; and
mold spores

105. The method of claim 56 wherein the robot further comprises an environmental sampling device, wherein the environmental sampling device takes a sample using at least one of the following sampling methods:
swab sampling;
sponge sampling;
direct surface sampling;
air sampling.

106. The method of claim 105 wherein the sampling method is capable of detecting at least one of the following indicators of contaminated air or surfaces:
aerobic plate count;
psychotrophic plate count;
Enterobacteriaceae;
coliform;
yeast;
mold;
adenosine triphosphate (ATP).

107. The method of claim 56 wherein the robot further comprises a control system.

108. The method of claim 56 further comprising using the robot in a hospital.

109. The method of claim 56 further comprising using the robot in a storage facility.

110. The method of claim 56 further comprising using the robot in a civil defense shelter.
